(19) United States
(12) Patent Application Publication
(10) Pub. No.: US 2026/0273312 A1
(43) Pub. Date: Sep. 17, 2026

(54) FOCUSED ULTRASOUND STIMULATION FOR RENAL DENERVATION

(71) Applicant: Medtronic Ireland Manufacturing Unlimited Company, Dublin 2 (IE)

(72) Inventors: Binit Panda, Maple Grove, MN (US); Gerry O. McCaffrey, Galway (IE)

(73) Assignee: Medtronic Ireland Manufacturing Unlimited Company, Dublin 2 (IE)

(21) Appl. No.: 19/471,252

(22) PCT Filed: Mar. 20, 2024
(86) PCT No.: PCT/EP2024/057382

§ 371 Date: Sep. 30, 2025

Related U.S. Application Data
(60) Provisional application No. 63/455,703, filed on Mar. 30, 2023.

(51) Int. Cl. A61N 7/02; A61N 7/00

(52) U.S. Cl. CPC A61N 7/022; A61N 2007/0026; A61N 2007/003; A61N 2007/0043

(57) ABSTRACT

Systems and methods of performing a therapeutic procedure by applying a first focused ultrasound (FUS) stimulation energy to a blood vessel wall, observing a patient parameter in response to the first stimulation energy, applying a therapy to the blood vessel wall, applying a second FUS stimulation energy to the blood vessel, observing the patient parameter in response to the second FUS stimulation energy, and determining whether the therapy has been successful based on the observed patient parameter in response to the second FUS stimulation energy.

### CROSS-REFERENCE TO RELATED APPLICATION

This disclosure claims the benefit of, and priority to, U.S. Provisional Patent Application Ser. No. 63/455,703, filed on Mar. 30, 2023, the entire content of which is hereby incorporated by reference herein.

### TECHNICAL FIELD

This disclosure relates to systems and methods enabling positioning a therapeutic device within luminal tissues to enhance ablation during a therapeutic procedure.

### BACKGROUND

Catheters have been proposed for use with various medical procedures. For example, a catheter can be configured to deliver neuromodulation (e.g., denervation) therapy to a target tissue site to modify the activity of nerves at or near the target tissue site. The nerves can be, for example, sympathetic or parasympathetic nerves. The sympathetic nervous system (SNS) is a primarily involuntary bodily control system typically associated with stress responses. Chronic over-activation of the SNS is a maladaptive response that can drive the progression of many disease states. For example, excessive activation of the renal SNS has been identified experimentally and in humans as a likely contributor to the complex pathophysiology of arrhythmias, hypertension, states of volume overload (e.g., heart failure), and progressive renal disease.

Percutaneous renal denervation is a minimally invasive procedure that can be used to treat hypertension and other diseases caused by over-activation of the SNS. During a renal denervation procedure, a clinician delivers stimuli or energy, such as radiofrequency, ultrasound, cooling, or other energy to a treatment site to reduce activity of nerves surrounding a blood vessel. The stimuli or energy delivered to the treatment site may provide various therapeutic effects through alteration of sympathetic nerve activity.

During current denervation procedures, it is not possible for a clinician to visualize the nerves prior to application of the therapy. Instead, denervation catheters are positioned to the best of the clinician's abilities and several ablations performed. As a result, the clinicians have no indication the ablations they are performing are actually ablating any nerves. Further, there is no indication during the procedure that the ablation was successful. Accordingly, this disclosure is directed to systems and methods of addressing these shortcomings of the current technologies.

### SUMMARY

One aspect of the disclosure is directed to a method of performing a therapeutic procedure. The method includes applying a first focused ultrasound (FUS) stimulation energy to tissue in or proximate a blood vessel wall. The method also includes observing a patient parameter in response to the first stimulation energy. The method also includes applying a therapy to the blood vessel wall. The method also includes applying a second FUS stimulation energy to the blood vessel wall. The method also includes observing the patient parameter in response to the second FUS stimulation energy. The method also includes determining whether the therapy has been successful based on the observed patient parameter in response to the second FUS stimulation energy. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method where the patient parameter includes at least one of blood pressure, pulse wave velocity (PWV) within a blood vessel associated with the blood vessel wall, blood vessel diameter, or electrically evoked compound action potential (ECAP) from a nerve in or proximate the blood vessel wall. The first FUS stimulation energy and the second FUS stimulation energy are applied to sympathetic nerves in or proximate the blood vessel wall. The blood vessel is a hepatic or a renal artery. The method further including determining whether the at least one of blood pressure, PWV, or ECAP observed following the application of the second FUS stimulation energy is less than the at least one of blood pressure, PWV, or ECAP observed following the first FUS stimulation energy. If the change is greater than the threshold the therapy is determined to be successful. The method further including navigating a therapy device to a location within an artery of the patient. The method further including laparoscopically placing a FUS energy delivery device extra-vascularly proximate the blood vessel wall. The method further including applying FUS energy from outside a body of a patient to a nerve in or proximate the blood vessel wall. A PWV in excess of a threshold is indicative a patient that is a candidate for a denervation procedure. The first FUS stimulation energy and the second FUS stimulation energy are part of a sequential session, an alternating session, or a simultaneous session. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A further aspect of the disclosure is directed to a system for denervation of nerves of a blood vessel. The system includes a catheter configured for navigation within a blood vessel of a patient. The system also includes at least one energy element formed on a distal portion of the catheter. The system also includes a sensor configured to measure one or more patient parameters operably connected to the catheter. The system also includes a focused ultrasound (FUS) stimulation energy source operably connected to the catheter. The system also includes a therapeutic energy source configured to apply therapy to the blood vessel. The system also includes a computing device including a memory and a processor and storing thereon instructions that when executed cause the computing device to: cause the FUS stimulation energy source to generate a first FUS stimulation energy for application to a blood vessel wall via the at least one energy element, cause the sensor to sense a first change in a patient parameter as a result of the application of the first FUS stimulation energy to the blood vessel wall, cause the therapeutic energy source to generate a therapeutic energy for application to or proximate the blood vessel, cause the FUS stimulation energy source to generate a second FUS stimulation energy for application to the blood vessel wall via the at least one energy elements, cause the sensor to sense a second change in the patient parameter as result of the application of the second FUS stimulation energy to the blood vessel wall, and determine whether the therapy has been successful based on the sensed second change in the patient parameter in response to the second FUS stimulation energy. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The system where the patient parameter includes at least one of blood pressure, pulse wave velocity (PWV) within the blood vessel, blood vessel diameter, or electrically evoked compound action potential (ECAP) from a nerve in or proximate the blood vessel wall. The memory stores thereon instructions that when executed by the processor cause the computing device to determine whether at least one of blood pressure, PWV, or ECAP observed following the application of the second FUS stimulation energy is less than at least one of the blood pressure, PWV, or ECAP observed following the first FUS stimulation energy. The memory stores thereon instructions that when executed by the processor cause the computing device to determine whether a change in blood pressure, PWV, blood vessel diameter, or ECAP between the first FUS stimulation energy and the second FUS stimulation is greater than a threshold, where if the change is greater than the threshold the therapy is successful. The generated first ultrasound stimulation energy and the generated second ultrasound stimulation energy are part of a sequential session, an alternating session, or a simultaneous session. The therapeutic energy source is a focused ultrasound energy source in communication with one of the at least one energy element formed on a distal portion of the catheter. The system further including an external focused ultrasound transducer operably connected to the therapeutic energy source and configured to apply high intensity focused ultrasound energy to or proximate to the blood vessel. The first FUS stimulation energy and the second FUS stimulation energy are applied to sympathetic nerves in or proximate the blood vessel wall. The blood vessel is a hepatic or a renal artery. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Further disclosed herein are systems and methods of performing a therapeutic procedure by applying a first focused ultrasound (FUS) stimulation energy to a blood vessel wall, observing a patient parameter in response to the first stimulation energy, applying a therapy to the blood vessel wall, applying a second FUS stimulation energy to the blood vessel, observing the patient parameter in response to the second FUS stimulation energy, and determining whether the therapy has been successful based on the observed patient parameter in response to the second FUS stimulation energy.

### BRIEF DESCRIPTION OF THE DRAWINGS

Various aspects and embodiments of the disclosure are described hereinbelow with references to the drawings, wherein:

FIG. 1 is a schematic diagram of a therapy system provided in accordance with the disclosure;

FIG. 2 is a schematic view of a workstation of the therapy system of FIG. 1;

FIG. 3 is a perspective view of a therapeutic device of the therapy system of FIG. 1 advanced within a portion of the patient's anatomy and in a deployed condition in accordance with the disclosure;

FIG. 4A is a graphical representation of changes in physiological parameters experienced by a patient as a result of a pre-therapy application of stimulation in accordance with the disclosure;

FIG. 4B is a graphical representation of changes in physiological parameters experienced by the patient as a result of post-therapy application of stimulation in accordance with the disclosure;

FIG. 5A is a representation of an external stimulation energy transducer in accordance with the disclosure;

FIG. 5B is a representation of an internal stimulation energy transducer in accordance with the disclosure;

FIG. 6A is a representation of a sequential therapy session;

FIG. 6B is a representation of an alternating therapy session;

FIG. 7 is a representation of a simultaneous therapy session;

FIG. 8 is a flow chart detailing a method of applying stimulation and therapy in accordance with the disclosure;

FIG. 9 is a method of applying stimulation and therapy in accordance with the disclosure;

FIG. 10 is a method of applying stimulation and therapy in accordance with the disclosure.

FIG. 11 depicts a device for stimulation and detection of electrically evoked compound action potential and denervation of a nerve in accordance with the disclosure;

FIG. 12 depicts a device for stimulation and detection of electrically evoked compound action potential and denervation of a nerve in accordance with the disclosure;

FIG. 13A is a visual representation of blood vessels before applying stimulation and therapy in accordance with the disclosure;

FIG. 13B is a visual representation of blood vessels after applying stimulation and before applying therapy in accordance with the disclosure;

FIG. 13C is a visual representation of blood vessels after applying therapy in accordance with the disclosure; and

FIG. 13D is a visual representation of blood vessels after applying stimulation and therapy in accordance with the disclosure.

### DETAILED DESCRIPTION

This disclosure is directed to therapeutic systems and methods for denervation or neuromodulation of nerves such as the sympathetic, or parasympathetic, nerves, and in particular, unmyelinated nerve fibers in and around blood vessels and other luminal tissues. Further, this disclosure is directed to systems and methods that provide pre-procedure guidance as to proper placement of a therapy catheter, intraprocedural guidance on the effects of the therapy, and therapy end point determination.

For ease of description, much of the following description focuses on implementations of focused ultrasound (FUS) stimulation in combination with ultrasound (US) denervation or radio frequency (RF) denervation. Those having skill in the art will recognize that the methods and systems described herein may employ other stimulation or therapy modalities including without limitation microwave, FUS, cryotherapy, chemical, and other modalities without departing from the scope of the disclosure. Similarly, the following description focuses on navigation to and application of neurostimulation and/or therapy to the renal artery to denervate sympathetic or, in certain embodiments, parasympathetic, nerves in, around, and proximate the renal arteries. However, the present disclosure is not so limited and can be employed for denervating nerves accessible via any blood vessel described herein (e.g., celiac trunk, hepatic, splenic, gastric, superior mesenteric, inferior mesenteric, gonadal, splanchnic, etc., and branches and/or combinations of each) of other luminal tissue (e.g., a bile duct).

FUS is an effective modality for stimulating neuronal activity, both in the central nervous system and in the peripheral nervous system. In practice, FUS activates mechanosensitive ion channels within the nerves. In contrast to electrical stimulation of axons, which activate a voltage gated ion channels in the nodes of Ranvier to generate an action potential, FUS stimulation is fundamentally a mechanical force (akin to use of a reflex hammer) to trigger a response. One aspect of the disclosure is directed to the use of FUS to stimulate neuronal response to assess when an end point for therapy has been reached.

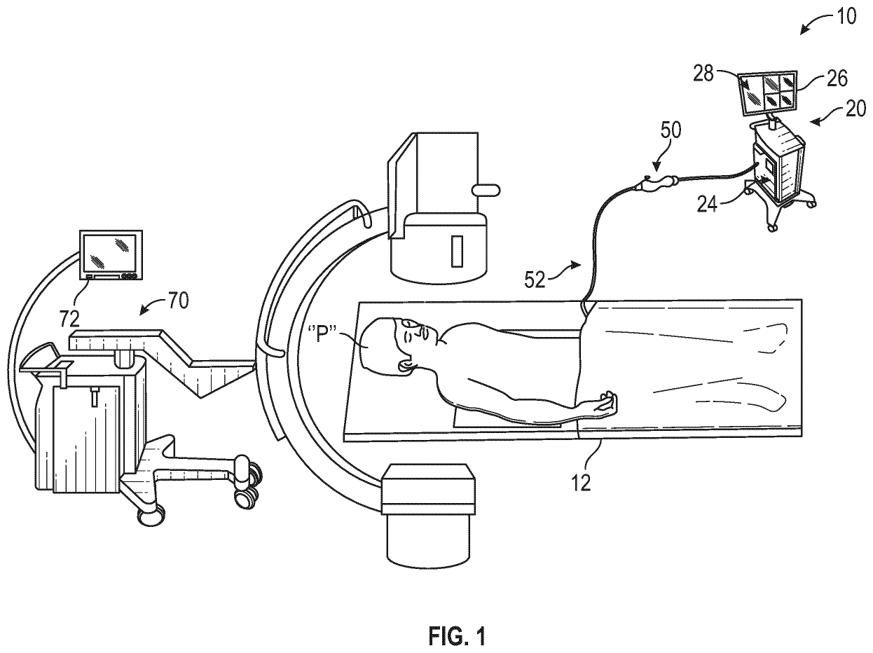

Turning now to the drawings, FIG. 1 illustrates a guidance and therapy system provided in accordance with the present disclosure and generally identified by reference numeral 10. As will be described in further detail hereinbelow, the guidance and therapy system 10 enables navigation of a therapeutic device 50 to a desired location within the patient's anatomy (e.g., the patient's renal artery), delivery of neurostimulation energy to tissue within the renal artery, observing a physiological response to the application of neurostimulation energy to the tissue, if necessary adjustment of a position of the therapeutic device within the renal artery based upon the physiological response, reapplication of the neurostimulation to the tissue at the adjusted position, application of denervation therapy energy to the tissue within the renal artery to denervate sympathetic nerves within the tissue, and delivery of neurostimulation energy to the denervated tissue observe the physiological response to the neurostimulation energy and assess the efficacy of the denervation therapy.

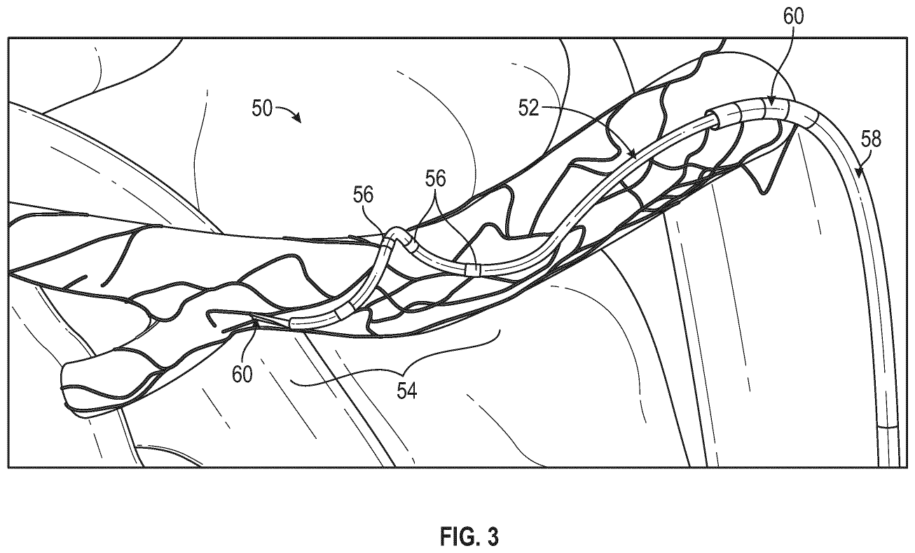

The guidance and therapy system 10 includes a workstation 20, a therapeutic device 50 operably coupled to the workstation 20, and an imaging device 70, which may be operably coupled to the workstation 20. The patient “P” is shown lying on an operating table 12 with the therapeutic device 50 inserted through a portion of the patient's femoral artery, although it is contemplated that the therapeutic device 50 may be inserted into any suitable portion of the patient's vascular network that is in fluid communication with a desired blood vessel for therapy. Although generally described as having one therapeutic device 50, it is envisioned that the therapy system 10 may employ any suitable number of therapeutic devices 50. The therapeutic devices 50 may employ the same or different therapy modalities may and be operably coupled to the workstation 20. Further, the therapeutic device 50 may employ a guidewire or a guide catheter 58 (FIG. 3) without departing from the scope of the disclosure.

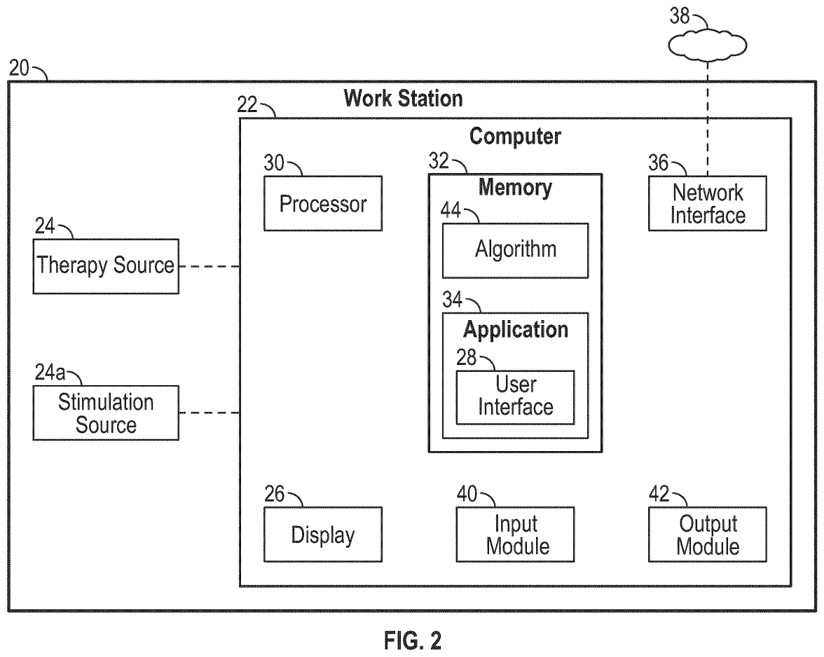

Continuing with FIG. 1 and with additional reference to FIG. 2, the workstation 20 includes a computer 22, a therapy energy source 24 (e.g., an ultrasound generator, RF generator, a microwave generator, a cryogenic medium source, a chemical source, etc.) operably coupled to the computer 22, and a stimulation energy source 24a operably coupled to the computer 22. Although generally described as being separate from the therapy energy source 24, it is envisioned that the stimulation energy source 24a may be integrated within the therapy source 24, and the therapy source 24 may generate the therapeutic energy and the stimulation energy modalities, for example where US is employed for therapeutic energy and FUS is employed for stimulation energy.

The computer is coupled to a display 26 that is configured to display one or more user interfaces 28. The computer 22 may be a desktop computer or a tower configuration with display 26 or may include a laptop computer or other computing device. The computer 22 includes a processor 30 which executes software stored in a memory 32. The memory 32 may store one or more applications 34 and/or algorithms 44 to be executed by the processor 30. A network interface 36 enables the workstation 20 to communicate with a variety of other devices and systems via the internet. The network interface 36 may connect the workstation 20 to the Internet via a wired or wireless connection. Additionally, or alternatively, the communication may be via an ad hoc Bluetooth® or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further data, image data, and/or videos may be stored. The cloud storage system 38 may be remote from or on the premises of the hospital such as in a control or hospital information technology room. It is envisioned that the cloud storage system 38 could also serve as a host for more robust analysis of acquired images (e.g., fluoroscopic, computed tomography (CT), magnetic resonance imaging (MRI), cone-beam computed tomography (CBCT), etc.), data, etc. (e.g., additional or reinforcement data for analysis and/or comparison). An input module 40 receives inputs from an input device such as a keyboard, a mouse, voice commands, an energy source controller (e.g., a foot pedal or handheld remote-control device that enables the clinician to initiate, terminate, and optionally, adjust various operational characteristics of the therapy energy source 24 or the stimulation energy source 24a, including, but not limited to, power delivery), amongst others. An output module 42 connects the processor 30 and the memory 32 to a variety of output devices such as the display 26. In embodiments, the display 26 may be a touchscreen display.

The therapy energy source 24 generates and outputs one or more of US energy, RF energy (monopolar or bipolar), microwave energy, cryogenic medium, or chemical ablation medium via an automated control algorithm 44 stored on the memory 32 and/or under the control of a clinician. As can be appreciated, the therapy energy generated or output by the therapy source 24 changes a temperature of the tissue (e.g., increases or decreased the temperature) to achieve the desired denervation of the nerves. The therapy energy source 24 may be configured to produce a selected modality and magnitude of energy or therapy for delivery to the treatment site via the therapeutic device 50, as will be described in further detail hereinbelow. The therapy energy source 24 may monitor relevant energy parameters (such as voltage and current in the context of ultrasound or RF energy) employed to generate the therapy energy applied to target tissue via the therapeutic device 50 and monitor the temperature of the target tissue or tissue proximate the target tissue, or a portion of the therapeutic device 50.

The stimulation energy source 24a generates a signal that is transmitted to a FUS transducer that generates a mechanical pulse of energy that is at a magnitude which will generally not impart therapy on the tissue. The stimulation energy generated by the stimulation energy source 24a does not denervate the target tissue. Rather, the stimulation energy source 24a generates a stimulation energy that can be applied to one or more nerves to stimulate the nerve and cause a physiological response that can indicate whether the patient is a candidate for denervation, whether a therapy device has been placed proximate nerves that are to receive therapy, assessed for an endpoint of the therapy, or whether successful denervation has been achieved. Responses to stimulation energy may include an increase in one or more of blood pressure, vessel stiffness, pulse wave velocity, augmentation pressure, heart rate variability, vasoconstriction, etc., and combinations of these depending on whether stimulation energy is applied. In accordance with the disclosure, the stimulation energy is FUS energy applied to the nerve.

FIG. 3 depicts one aspect of a therapeutic device 50 in accordance with the disclosure. The therapeutic device 50 includes an elongated shaft 52 having a handle (not shown) disposed on a proximal end portion of the elongated shaft 52. The therapeutic device 50 includes an energy delivery assembly 54 at which one or more energy elements 56 are located. The elongated shaft 52 of the therapeutic device 50 is configured to be advanced within a portion of the patient's vasculature, such as a femoral artery or other suitable portion of patient's vascular network that is in fluid communication with the patient's renal artery. In embodiments, the energy delivery assembly 54 is configured to be transformed from an initial, undeployed configuration having a generally linear profile, to a second, deployed or expanded configuration, where the energy delivery assembly 54 forms a radially expanded configuration, such as a generally spiral and/or helical configuration, for delivering energy to a site for either or both application of a stimulation signal and/or therapeutic energy at the treatment site. Those of skill in the art will recognize in the context of the instant application that application of therapeutic energy should be construed to include application of cryogenic cooling to the treatment site to achieve a thermally induced neuromodulation. In this manner, when in the second, expanded configuration, the energy delivery assembly 54, and in particular, the individual energy elements 56, is pressed against or otherwise contacts the walls of the patient's vasculature tissue. Although generally described as transitioning to a spiral and/or helical configuration, it is envisioned that the energy delivery assembly 54 may be deployed in other configurations (such as an expanded frame or basket, a balloon, or the like) without departing from the scope of the present disclosure. Further, the therapeutic device 50 may be configurable, for example, using one or more pull wires or other control mechanisms (not shown) to adjust the configuration to promote contact between the energy elements 56 and the wall of the renal artery. As such, the therapeutic device 50 may be capable of being placed in one, two, three, four, or more different configurations depending upon the design needs of the therapeutic device 50 or the location at which therapy is to be applied.

As depicted in FIG. 3, the elongated shaft 52 may be configured to be received within a portion of a guide catheter or guide sheath (such as a 6F guide catheter) 58 that is utilized to navigate the therapeutic device 50 to a desired location at which point if a guide catheter 58 is retracted to uncover the therapeutic device 50. As noted hereinabove, retraction of the guide catheter 58 may enable the energy delivery assembly 54 to transition from the first, undeployed configuration, to the second, deployed or expanded configuration.

The elongated shaft 52 of the therapeutic device 50 may further include an aperture (not shown) at a distal end thereof and configured to slidably receive a guidewire over which the therapeutic device 50, either alone or in combination with the guide catheter 58, are advanced. In this manner, the guidewire is utilized to guide the therapeutic device 50 to the target tissue using over-the-wire (OTW) or rapid exchange (RX) techniques, at which point the guide wire may be partially or fully removed from the therapeutic device 50, enabling the therapeutic device 50 to transition from the first, undeployed configuration, to the second, deployed or expanded configuration (FIG. 3). As noted elsewhere herein, the therapeutic device 50 may be transition from the first, undeployed configuration to the second, deployed configuration automatically (e.g., via a shape memory alloy, etc.) or manually (e.g., via pull wires, guide wire manipulation, etc. that is controlled by the clinician).

Continuing with FIG. 3, the energy elements 56 are disposed on an outer surface of the elongate shaft 52 and configured to contact a portion of the patient's vascular tissue when the therapeutic device 50 is placed in the second, expanded configuration. As shown herein, the therapeutic device 50 includes four energy elements 56. However, the present disclosure is not so limited and the therapeutic device 50 may have more or fewer energy elements 56 without departing from the scope of the present disclosure. One of skill in the art will recognize that the energy elements 56 may be one or more of ultrasound transducers, RF electrodes, microwave antennae, ports for delivery of cryoablation medium or chemical medium and other implements and/or ablation and denervation modalities without departing from the scope of the present disclosure. Further, the energy elements 56 may be combined mode energy elements enabling the same energy element 56 to apply FUS stimulation energy and also US, RF, microwave, or other therapeutic energy to the blood vessel wall.

As illustrated in the figures, the energy elements 56 may be disposed in spaced relation to one another along a length of the therapeutic device 50 forming the energy delivery assembly 54. As will be appreciated, these energy elements 56 may be in communication with one or both the therapy energy source 24 and the stimulation energy source 24a. It is envisioned in one embodiment that the therapy energy source 24 is also the stimulation energy source 24a and includes a diagnostic mode, where the therapy energy source 24 generates a stimulation signal, and a denervation mode, where the therapy energy source 24 generates therapy signals to denervate the nerves of the relevant blood vessel. It is contemplated that the therapy energy source 24 may be manually switched from a stimulation mode to a denervation mode and vice versa or may be automatically switched by an algorithm 44 stored on the memory 32 of the computing device. Alternatively, the energy elements 56 are in communication with a stand-alone stimulation energy source 24a to deliver a stimulation signal to the blood vessel in question. The stimulation energy is generated by the stimulation energy source 24a and communicated to the energy elements 56 causing stimulation of the sympathetic nerves as described herein.

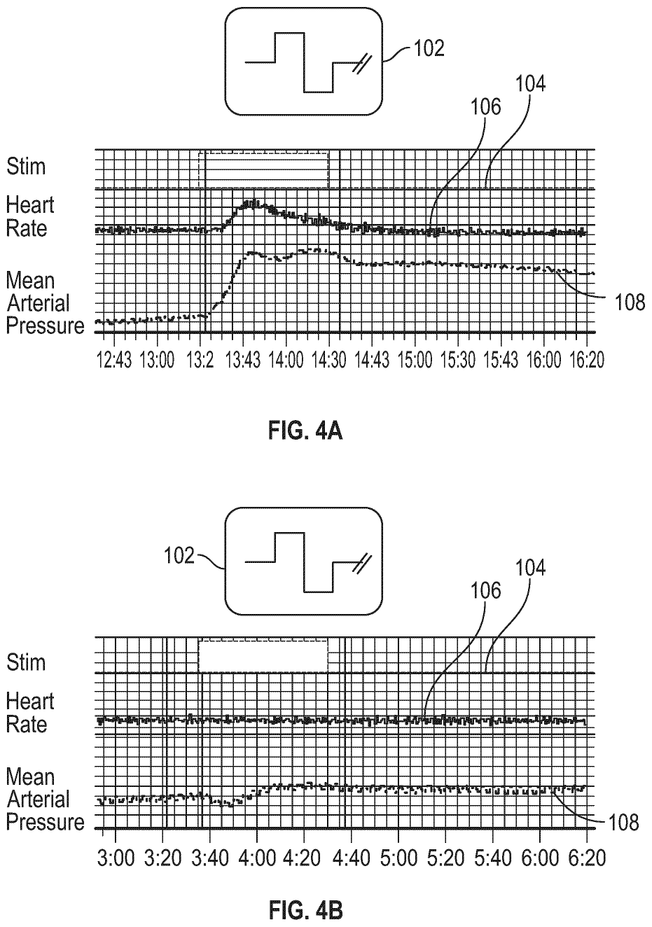

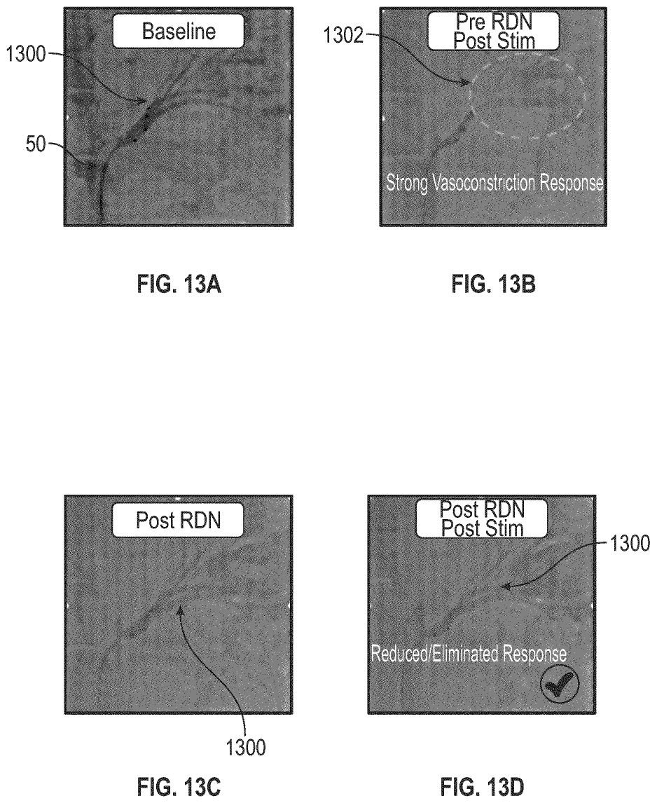

FIGS. 4A and 4B depict one aspect of the disclosure. As depicted in FIG. 4A, a stimulation signal 102 is applied via the energy elements 56 to the target tissue. The duration of the stimulation signal is graphically depicted by trace 104. As a result of the stimulation signal 102, during its application two physiological effects are observed. The first effect is an increase in heart rate as depicted by trace 106. As can be seen, even before cessation of the stimulation signal 102, the heart rate begins to return to normal. Conversely, the mean arterial pressure, as depicted in trace 108, both during and following the application of the stimulation signal 102 elevates and remains elevated compared to the pre-stimulation mean arterial pressure. This change in either or both heart rate and mean arterial blood pressure is indicative of stimulation of the afferent nerves of the blood vessel in which the therapeutic device 50 is positioned (e.g., the renal and/or hepatic arteries). In instances where the change in heart rate or mean arterial pressure is in excess of a predetermined threshold, the clinician may determine that the location of the energy delivery assembly 54 is appropriate for application for therapy to achieve denervation and therapeutic energy may be applied to the target tissue at that location within the blood vessel. With additional reference to FIG. 13A-13D, it is envisioned that a third physiological effect resulting from the application of the stimulation signal 102, such as vasoconstriction, may be observed. As will be described in further detail hereinbelow, vasoconstriction, in which the application of the stimulation signal 102 effectuates a reduction in blood vessel diameter at and around the target tissue, may be visually identified using external or internal imaging modalities, and in instances where there is a change in vessel diameter that is in excess of a predetermined threshold, the clinician may determine that the location of the energy delivery assembly 54 is appropriate for application for therapy to achieve denervation and therapeutic energy may be applied to the target tissue at that location within the blood vessel.

In one aspect of the disclosure, as depicted in FIG. 4B following application of the therapeutic energy, the stimulation signal 102 may again be applied as shown in trace 104. As shown, despite the application of the stimulation signal 102, very little response is observed in either heat rate 106, in mean arterial pressure 108 in FIG. 4B, or in diameter of blood vessels within or around the location where the stimulation signal 102 is applied. The difference between the observed response to the stimulation signal 102 applied before therapy (FIG. 4A) and the observed response to the stimulation signal 102 applied after therapy (FIG. 4B) is indicative of a successful denervation of the afferent nerves (e.g., sympathetic, or parasympathetic nerves) proximate the placement of the therapeutic device 50 within the blood vessel. Thus, employing the systems and methods of this disclosure a clinician can navigate a therapeutic device 50 to a location within the patient P, apply a stimulation signal 102 to confirm that the therapeutic device 50 is placed proximate afferent nerves, apply a therapy to the nerves, and apply stimulation a second time to confirm the successful denervation of the nerves or to determine that further application of therapy is needed. As noted above, this cycle may be repeated as needed to achieve a successful ablation. In accordance with this disclosure, the stimulation signal may be FUS energy. As described elsewhere herein, a therapy energy source 24 and a stimulation energy source 24a apply signals to energy elements 56 of the therapeutic device 50. In the case of the energy elements 56 being RF electrodes, as therapy is applied to the energy elements 56, and there with the target tissue in an effort to denervate the afferent nerves located therein the impedance of the tissue through which the energy is passed can be measured by the therapy source 24. Further, regardless of the modality of the energy elements 56 as energy is applied to the tissue, here the blood vessel wall, the tissue will begin to heat. However, it is desirable to prevent the tissue from exceeding a predetermined temperature at which permanent damage to the tissue of the blood vessel wall occurs. Fortunately, nervous tissue tends to be more susceptible to heat and denatures or allows for denervation at temperatures below that at which the surrounding tissue experiences permanent and irreversible damage. Thus, by monitoring the temperature of the target tissue, the therapy source 24 can be controlled to prevent undesirable heating of the blood vessel. In one aspect of the disclosure each energy element 56 may incorporate a thermistor or other temperature sensor (not shown) to monitor the temperature of the energy element 56. As will be appreciated, the energy elements 56 directly contact the inner wall of a blood vessel or other luminal tissues, thus as energy is passed through the energy elements 56, the energy elements 56 themselves begin to heat. The thermistor, thermocouple or other temperature senor in communication with the energy element 56, generates a signal that is received by the therapy energy source 24. That signal is representative of the temperature of the energy element 56, if that temperature of any of the energy element 56 exceeds a predetermined threshold, below that at which damage might occur to the blood vessel wall, the therapy energy source 24 stops outputting therapeutic energy to the energy element 56. As will be appreciated, the movement of blood through the blood vessel will cool the energy element 56. Once the temperature of the energy element 56 returns below a predetermined threshold, the therapy energy source 24 may again begin applying therapy using one or more of the methods descried herein to achieve the desired denervation or neuromodulation of the nerves surrounding the blood vessel.

In addition to the above-described forms of feedback which are primarily, though not exclusively, employed to protect the patient and the tissues of the patient during the procedure, another form of feedback may be provided via a blood pressure module. A blood pressure module 62 employing a blood pressure sensor 60 located on the therapeutic device 50, catheter 58 or a separate component navigated proximate the target tissue. In either event, the blood pressure sensor 60 monitors the blood pressure in the blood vessel to which therapy is applied. That measured blood pressure may be used directly as the feedback parameter or may be converted to one or more different indicia including but not limited to pulse wave velocity, augmentation index (AIX) a measure of arterial stiffness, or tricuspid regurgitation velocity (TR).

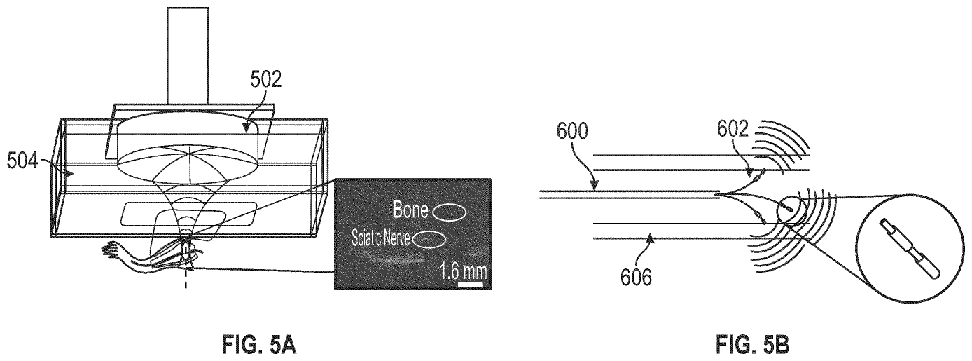

FIG. 3 depicts a therapeutic device 50 that can apply the FUS neurostimulation signal, as well as the therapy (e.g., US or RF energy). However, the instant disclosure is not so limited. Indeed, FIG. 5A depicts a HIFU transducer 502, which is acoustically coupled to a patient via a coupling medium (e.g., saline) in a coupling chamber 504 that is placed on the skin of the patient. Alternatively, in place of the coupling medium in a coupling chamber 504, a gel material acoustically coupling the HIFU transducer 502 with the patient may be employed without departing from the scope of the disclosure. The entire HIFU transducer 502 remains external to the patient. The HIFU transducer 502 can be employed to both image portions of the patient, apply FUS neuro stimulation signals, and to apply US therapy signals to the sympathetic nerves of an identified artery. Alternatively, as shown in FIG. 5B, the neurostimulation catheter 600 may be employed. The catheter 600 may have one or more HIFU transducers 602 and be configured to be navigated within a patient's arteries to apply stimulation FUS energy to the nerves surrounding the blood vessel 606 in accordance with aspects of the disclosure. A third possibility is a laparoscopic approach where the HIFU transducer, e.g., similar to that shown in FIG. 5B is placed substantially non-invasively proximate the nerves to be stimulated or denervated (e.g., the nerves surrounding the renal or hepatic nerves). In such instances, a separate therapy device for application of a therapy to the nerves (e.g., the therapy device 50 of FIG. 3) may be required for the application of therapeutic US or RF energy to perform the denervation. In embodiments, the one or more HIFU transducers 602 can be employed to both image portions of the blood vessels from an internal perspective, apply FUS neuro-stimulation signals, and to apply US therapy signals to the sympathetic nerves of an identified artery.

As noted above, the amplitude, frequency, duty cycle, pulse width, or duration of the neurostimulation energy or can be selected or modified to ensure neurostimulation of the sympathetic nerves of the luminal tissue without damaging the luminal tissue or the nerves within or surrounding the luminal tissue. In one non-limiting embodiment, the stimulation source 24a is connected to a High Intensity Focused Ultrasound transducer to produce a FUS energy at a frequency of between approximately 250 KHz and 700 KHz, though certain applications employing an external HIFU transducer can require up to 4 MHz, for example about 3.57 MHz. The duty cycle may range from about 5-about 10 % depending on the vessel wall composition. A therapy session (stimulation +therapy) may have a duration of between 30 and 120 seconds.

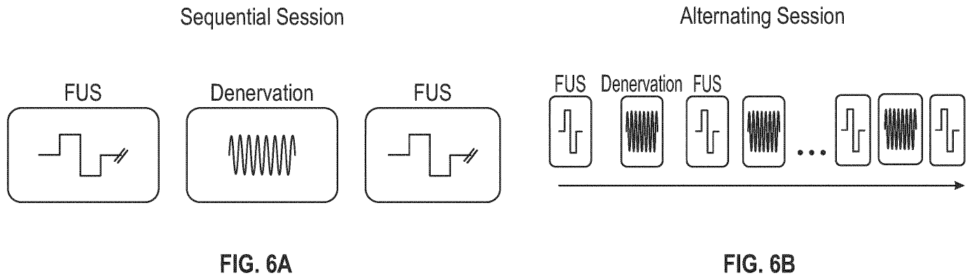

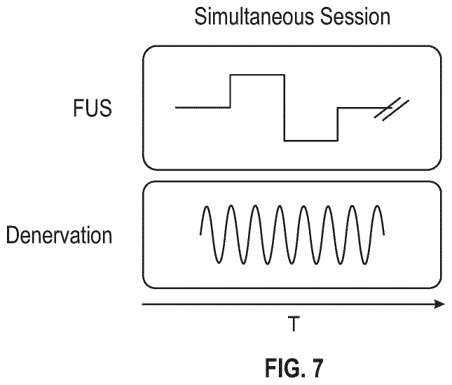

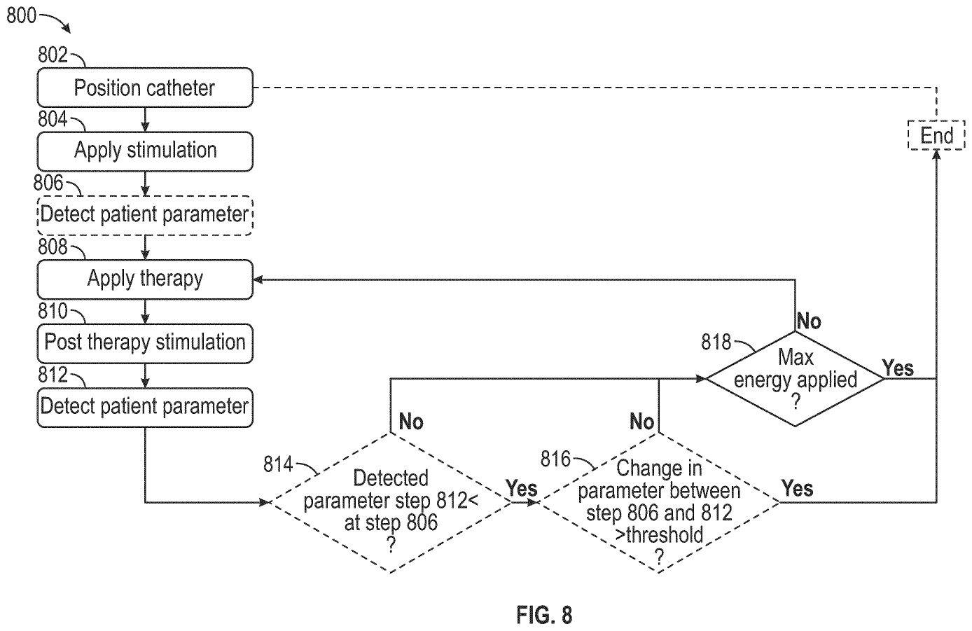

Exemplary therapy sessions can include sequential stimulation and denervation (FIG. 6A) or alternating stimulation and denervation (FIG. 6B) or simultaneous stimulation and denervation (FIG. 7). It is envisioned that in a sequential stimulation and denervation session (FIG. 6A) the FUS stimulation is applied to the tissue to achieve a response for between 5 and 60 seconds, in certain aspects about 10 seconds followed by application of therapy, for example ultrasound therapy, for a duration of between 20 and 60 seconds, in certain aspects about 40 seconds, and then a further of FUS stimulation for between 5 and 20 seconds, in certain aspects about 10 seconds. As will be described below in connection with FIG. 8, if there is no response or a substantially reduced response following the second FUS stimulation a successful denervation has been achieved and the session ends. This alternating between FUS stimulation and ultrasound therapy can be repeated until the response is below the threshold or until a maximum energy application value has been reached. Alternatively, the therapy may be reapplied and repeated until a FUS stimulation results in a nervous response less than a threshold. In an alternating stimulation and denervation embodiment (FIG. 6B) a series of shorter duration of FUS stimulation signals (e.g., between 5 and 20 ms, in certain aspects about 10 ms) are applied followed by therapy (e.g., ultrasound) of between 20 and 60 ms (in certain aspects about 40 ms). This pattern of, for example 10 ms of FUS stimulation followed by 40 ms of ultrasound therapy is repeated for an overall duration (e.g., between 30 and 120 seconds, in certain aspects about 60 seconds). With each application of the FUS stimulation, or a subset of the FUS stimulations, the nervous response can be observed. If following the last application of the FUS stimulation the response to the FUS stimulation is below a threshold, the denervation therapy has been successful. If the response to the final FUS stimulation is greater than the threshold, the session may be repeated, either for the full duration or some fraction of the original duration, until the response to the FUS stimulation is below the threshold or a maximum energy application value has been reached.

In the simultaneous stimulation and denervation, a FUS stimulation is applied for a short duration (e.g., 5-20 ms up to 1-5 s) prior to application of the therapy. The stimulation and the therapy are applied until either a change in for example measured blood pressure or pulse wave velocity are detected, a change in the response to the stimulation is detected, a change in vessel diameter is detected, or until an energy application threshold (e.g., duration, tissue temperature, energy quantity) is reached. In instances where a change in the measured parameter or a change in response to stimulation is not detected, the process may be repeated. During the short duration of only stimulation being applied, a determination can be made whether a response is detected, and if no response is detected, the application of the denervation energy may be stopped or prevented and one or more indicators may be presented to a user indicating either that the placement of the therapy device 50 should be adjusted, or that a preceding application of therapy has been successful.

In a similar fashion, FUS stimulation may be coupled with RF therapy, as either Sequential Sessions, Alternating Sessions, Simultaneous Sessions. In accordance with a sequential session, the FUS stimulation is applied for between 15 and 50 seconds, in certain embodiments about 30 seconds, followed by RF therapy of between about 30 and 120 seconds, in certain embodiments about 60 seconds. Again, a second FUS stimulation may be applied and where the nervous response is below a threshold, the denervation can be considered successful. If the response is above the threshold, the RF therapy can again be applied. This switching between FUS stimulation and RF therapy can be repeated until the response is below a threshold or until a maximum energy application value has been reached. Alternatively, in an alternating session an alternating series of FUS stimulation of between 5 and 20 seconds (in certain embodiments about 10 seconds), followed by application of RF therapy of between 5 and 20 seconds (in certain embodiments about 10 seconds). As above, with each application of the FUS stimulation, or a subset of the FUS stimulations, the nervous response can be observed. If following the last application of the FUS stimulation the response to the FUS stimulation is below a threshold, the denervation therapy has been successful. If the response to the final FUS stimulation is greater than the threshold, the session may be repeated, either for the full duration or some fraction of the original duration, until the response to the FUS stimulation is below the threshold or a maximum energy application value has been reached. Similarly, in a simultaneous session, the FUS stimulation is initiated for a short duration to trigger a stimulation response, and RF therapy is then initiated after the short duration. The RF therapy and FUS stimulation are simultaneously applied to the tissue until either the response to the FUS stimulation drops below a threshold, a measured parameter (e.g., blood pressure, pulse wave velocity, vessel diameter) drops below a threshold, or an RF therapy threshold is reached (e.g., duration, tissue temperature, energy quantity). As noted above this may be repeated, and during the application of the FUS stimulation, if no response is detected, the preceding application of the RF therapy can be considered successful.

As noted above, FUS can be employed, by altering the parameters of the drive signal supplied to the HIFU transducer, to both stimulate the nerves and to denervate the nerves to permanently inhibit neuronal activity. One of the mechanisms of actions of current denervation therapies is to permanently interrupt the neuronal activity along the sympathetic nerves of, for example, the renal or hepatic arteries. One of the challenges of known systems is determination of whether the patient is likely to benefit from the denervation procedure. Similarly, even if the patient is expected to benefit from the procedure, determination of the location of the sympathetic nerves remains challenging. Further, assessment of an endpoint for the denervation therapy presents yet another challenge. The use of FUS stimulation energy can be used to address all three of these concerns, and can provide benefits over electrical stimulation techniques, at least in part because the mechanism for triggering the neurological response is primarily mechanical.

The application of FUS stimulation energy to the sympathetic nerves in and around blood vessels, even those experiencing high blood pressure, nonetheless results in an increase in vessel stiffness (FIG. 4A), effectively a localized exacerbation of the stiffness. The effects may be measured by sensing in-vivo blood pressure within the artery to which FUS energy has been applied. Increases in blood pressure or increases in pulse wave velocity are both indications of increasing vessel stiffness following the application of the FUS stimulation. Alternatively, effects of the FUS stimulation energy can be observed through visualization of the renal or hepatic arteries (e.g., fluoroscopy or ultrasound) to measure a pulse wave velocity of blood through the renal or hepatic nerve, or a diameter of blood vessels at or around the application of the FUS stimulation. Thus, following application of FUS to stimulate neuronal activity in, for example, the renal or hepatic artery if there is an observed increase in blood pressure in that artery or a decrease in vessel diameter, the clinician can conclude that the patient is likely to benefit from denervation therapy.

Similarly, a detection of a change in blood pressure, pulse wave velocity, or vessel diameter by application of the FUS energy at a particular location along the artery wall indicates that the location is proximate sympathetic nerves and is a good location for application of therapy. Accordingly, in some aspects of the disclosure, through the use of imaging (e.g., fluoroscopy or ultrasound) a location at which the neuronal activity is stimulated can identified so that a treatment device is confirmed at or can be navigated to a location for the application of therapy. Similarly, if no change in blood pressure, pulse wave velocity, or vessel diameter is detected, then the clinician can alter the location of the application of the FUS energy and again assess the effects of the signal to identify a location or locations where the FUS energy inhibits the sympathetic nerve activity and thus a change in pulse wave velocity, blood pressure, or vessel diameter can be detected.

Further, by measuring the response to the FUS stimulation, determinations on an end point for the application of the therapy can also be determined. For example, by sensing a change in blood pressure or pulse wave velocity within the blood vessel, or a change in diameter of the blood vessel. Once a drop in excess of a threshold is observed, the therapy may be ceased. FIG. 8 depicts a flow chart showing a method 800 in accordance with the disclosure for application of a sequential therapy session. As noted above, the method 800 may follow method 700, or may be performed without performing method 700. The therapeutic device 50 described in connection with method 800 is capable of applying both FUS stimulation and either US denervation or RF denervation (or another modality denervation). The method may also be performed using separate FUS stimulation device (e.g., the external HIFU transducer 502) or a percutaneously inserted FUS stimulation device, without departing from the scope of the disclosure. With respect to method 800, at step 802, the therapeutic device 50 is placed at a desired location within the patient (e.g., in a renal or hepatic artery). As part of the placement, the therapeutic device 50 may be advanced from the catheter 58 and the therapeutic device 50 allowed to expand such that the energy elements 56 are in contact with an inner wall of the artery. At step 804 FUS stimulation energy is applied via the energy elements 56 to stimulate the nerves, particularly the sympathetic nerves that surround the blood vessel in which the therapeutic device 50 has been placed. The application of the FUS stimulation energy to the nerves (e.g., sympathetic nerves) surrounding the blood vessel causes the blood vessel to contract (e.g., for example, vasoconstriction). This contraction ensures that the energy elements 56 are in contact with the energy elements 56. As noted above, the FUS stimulation energy may be applied for example 30 seconds. At step 806 a change in a patient parameter (e.g., blood pressure, pulse wave velocity, vessel diameter, or others) may be optionally detected and the value stored in memory 32. In some instances, this detection can be employed to ensure that the placement of the energy elements 56 is indeed proximate the nerves to be denervated, and where no change in patient parameter is detected, an alert may be presented on the display 26 to inform the clinician that the therapeutic device 50 should be adjusted. At step 808 the therapy is applied to the blood vessel, and particularly to the sympathetic nerves surrounding the blood vessel. The therapy may be for example ultrasound, RF, or other modalities described herein. At step 810 following the application of the therapy, FUS stimulation energy is again applied, and at step 812 a change in patient parameter is detected. Following detection of the change in patient parameter at step 812 one or more optional steps may be undertaken. In one optional step, at step 814 the detected patient parameter at step 812 can be compared to the detected parameter at step 806 (e.g., blood pressure following the initial stimulation vs blood pressure following the second stimulation or vessel diameter following the initial stimulation vs vessel diameter following the second stimulation). If the parameter has not changed following the application of therapy at step 808 then denervation has not been completed and the process proceeds to step 818 where an assessment is made whether a maximum energy threshold has been applied to the blood vessel. If not, then the method returns to step 808 for application of more therapy, however, if the maximum energy threshold has been reached the session ends. If the answer is yes at step 814, or directly following step 812, the method may proceed to step 816 where a determination is made whether a change in the detected parameter between steps 806 and 812 is greater than a threshold. For example, if the blood pressure following step 812 were measured at 25 mm Hg less than that measured following step 806, that may be considered a successful denervation (i.e., an ability for the application of stimulation to stimulate the nerves surrounding the blood vessel). If no a step 816, the method may again proceed to step 818 for a determination of whether a maximum energy has been applied. As noted above, steps 814 and 816 are optional and may be individually employed or may be employed in various combinations without departing from the scope of the disclosure.

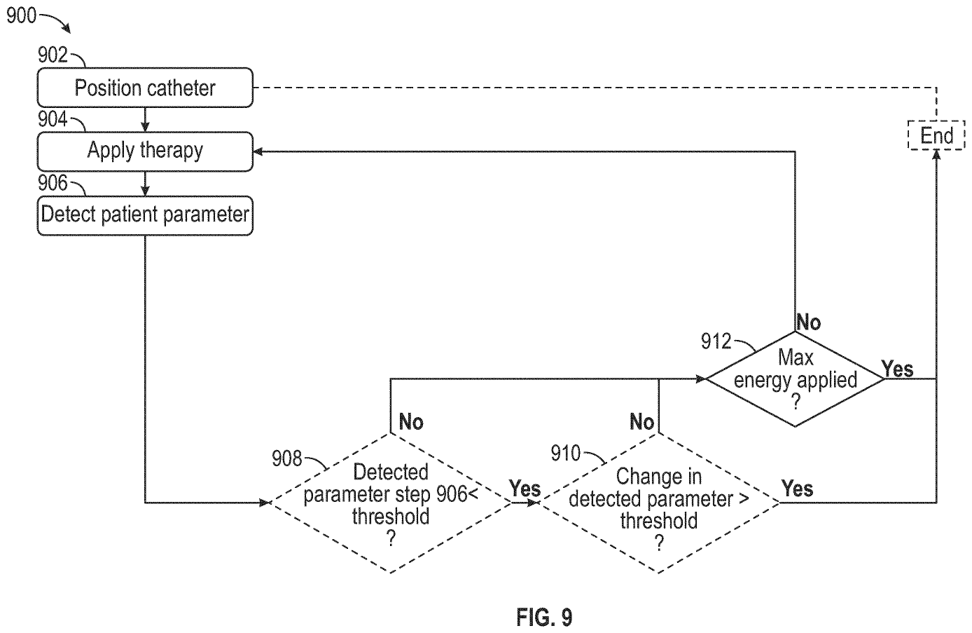

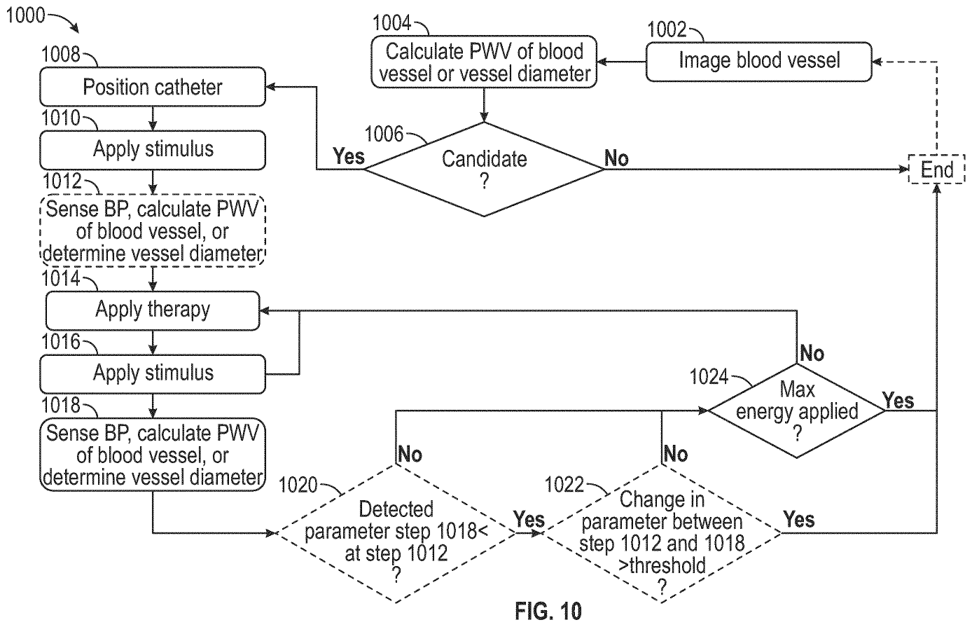

Method 800 is described above in connection with a sequential session. However, it is not so limited and an alternating session or a simultaneous session may also be employed for the application of the therapy without departing from the scope of the disclosure. However, in addition a similar method 900 may also be employed in connection with either an alternating or simultaneous session as shown in FIG. 9. As with method 800 the therapy device 50 is placed proximate the nerves to be denervated (e.g., within the renal or hepatic artery) at step 902. Next the simultaneous application of FUS stimulation energy and denervation energy (ultrasound, RF, or others) is applied to the wall of the blood vessel in question at step 904. Following a duration (e.g., 60 seconds) a patient parameter can be detected at step 906. Following detection of the patient parameter at step 906, one or more of steps 908 and 910 may be undertaken to assess the efficacy of the denervation and to determine whether the session should end. At step 908 the detected parameter is compared to a threshold. For example, a detected blood pressure or vessel diameter within the blood vessel can be compared to a healthy blood pressure or vessel diameter value. Alternatively, a change in the patient parameter may be detected as a result of the application of the therapy at step 904. This may be achieved by sensing blood pressure or visualizing vessel diameter before application of the therapy. That detected parameter may then be compared at step 910 to the detected blood pressure or visualized vessel diameter at step 906 to determine whether a therapy has been completed. If following either step 908 or 910 the answer is no, the method proceeds to step 912 for a determination of whether maximum energy has been applied. If yes at step 912 to method ends, if no at step 912 the method returns to step 904 for the application of more therapy. As an additional or alternative step, the comparison at step 910 may look to determine whether there is any detected change is trending in the correct direction (e.g., downward) for blood pressure or PWV, vessel diameter, or other patient parameter. Thus in at least one aspect of the disclosure, even if it is determined that the maximum energy has been applied (step 912) the therapy may be determined to be successful when the trend of the detected patient parameter is trending in the correct direction for that patient parameter.

In one example, employing a simultaneous FUS stimulation and therapy (FIG. 7) steps 904, 906, and 910 may occur simultaneously, with the end point for the therapy (step 910) being determined simultaneously with the application of stimulation and the application of the therapy.

The use of greater or less than in steps 814-816 and 908-910 are exemplary and should be considered to encompass embodiments where greater than or equal to or less than or equal to are utilized.

Yet a further aspect of the disclosure is directed to a method 1000 employing imaging. There have been developed methods of calculation of pulse wave velocity (PWV) of blood flowing through a blood from images of the blood vessel. Using imaging, the distension of the blood vessel as a pulse of blood (corresponding to a pulse ejected from the left ventricle of the heart) can be observed traversing along a length of the blood vessel. By defining a distance and determining the time required for the observable distension of the blood vessel to traverse the known distance, PWV can be calculated. PWV can therefore be used to identify potential candidates for denervation therapy. As will be appreciated, and as noted herein above, hypertension patients that benefit from denervation therapy exhibit enhanced blood vessel stiffness which correlate to increased blood pressure and PWV as a result of the increased stiffness. Accordingly, a potential patient that has a PWV in the hepatic or renal arteries above a predetermined level may be considered a candidate for a denervation procedure. In embodiments, using imaging, the diameter of the blood vessels after an application of neurostimulation can be observed and used to identify potential candidates for denervation therapy.

Method 1000 begins at step 1002 with imaging of a blood vessel such as a renal or hepatic artery. With the images captured, at step 1004 they are analyzed to calculate a PWV in the imaged blood vessel and additionally or alternatively, the captured images are analyzed to determine a diameter of one or more blood vessels along a length of the one or more imaged blood vessels. At step 1006 a determination is made whether the PWV indicates that the patient is a candidate for a denervation procedure, if the PWV is sufficiently low such that the patient is not considered a candidate the procedure ends. If, however, the PWV is sufficiently high that the patient is considered a candidate, the method moves to step 1008 where a therapy device 50 is positioned within the patient, for example, within the renal or hepatic artery. At optional step 1010, depending on which type of session is being employed a stimulus (e.g., FUS neurostimulation) is applied the wall of the blood vessel to stimulate the nerves of the blood vessel. At optional step 1012 following application of stimulation at step 1010, the blood pressure within the vessel can be detected using the blood pressure sensor 60 and additionally or alternatively, based on the sensed blood pressure a PWV can also be calculated and additionally or alternatively, the blood vessels may be imaged and the diameter of the one or more blood vessels may be determined.

At step 1014 therapy is applied via the energy elements 56 to the wall of the blood vessel in which the therapy device 50 has been positioned. This therapy may be either the sequential session (FIG. 6A) or the alternating session (FIG. 6B), or the sequential session (FIG. 7). In the case of alternating or sequential sessions step 1014 may be immediately after step 1008. As with other embodiments the therapeutic energy may be ultrasound, RF, microwave, or other modalities described herein. At step 1016 stimulus (e.g., FUS neurostimulation) is again applied to the blood vessel wall, and to the nerves, particularly the sympathetic nerves, that extend in or along the blood vessels. As will be appreciated, for alternating sessions (FIG. 6B), steps 1014 and 1016 may repeat until the desired duration is complete. Further for a sequential session (FIG. 7) steps 1014 and 1016 may occur substantially simultaneously. Following stimulation, at step 1018 the blood pressure within the vessel can again be detected using the blood pressure sensor 60 and additionally or alternatively, based on the sensed blood pressure a PWV can also be calculated, and additionally or alternatively, the blood vessels can be imaged and the diameter of the blood vessels can be determined. For a simultaneous session (FIG. 7) step 1020 may occur simultaneously with steps 1014 and 1016.

Following the blood pressure detection, PWV calculation, or blood vessel diameter determination at step 1018 one or more comparisons can be undertaken to determine whether therapy is complete. At step 1020 the blood pressure measured (or PWV) at step 1018 is less than the blood pressure (or PWV) at step 1010. For a simultaneous session (FIG. 7) step 1020 may occur simultaneously with steps 1014, 1016, and 1018. If the answer is no, the method proceeds to step 1024 where a determination is made whether a maximum amount of energy has been applied to the blood vessel. If yes at step 1024, method 1000 ends, if not then the method returns to step 1014 where therapy is again applied or in the case of the simultaneous session (FIG. 7) continues to be applied.

If yes at step 1020 or immediately following step 1018 the method progresses to step 1022 where the change in blood pressure, PWV, or diameter of the blood vessels between steps 1012 and 1018 is compared to a threshold. The threshold may be a fixed value e.g., 25 mm Hg, or a percentage change. If a simultaneous session (FIG. 7) is employed, the difference can be between a blood pressure, PWV, or blood vessel diameter calculated near the beginning of the simultaneous stimulation and therapy, and a blood pressure, PWV, or blood vessel diameter at the conclusion of the simultaneous application of energy. If the change exceeds the threshold, the method may be considered successful and the method 1000 ends. If the change is not in excess of the threshold the method returns again to step 1024 for a determination of whether the therapy applied exceeds a maximum energy value. If yes at step 1024 the method ends, but if not then the method returns to step 1014 for application of additional therapy.

The therapeutic devices 50 contemplated in this disclosure can apply one or more of a variety of therapeutic modalities. For example, the therapeutic modalities considered within the scope of this disclosure include monopolar or bipolar radiofrequency, microwave, cryogenic, ultrasound, chemical, and other yet to be developed modalities. Any of these therapy modalities may be incorporated into a therapeutic device, such as a catheter, which is configured for navigation to a desired location within the patient. A catheter configured to delivery one or more of these therapeutic modalities may be percutaneously navigated, for example via the femoral artery, to reach the blood vessels of the aorta including the celiac artery, hepatic arteries, splanchnic arteries, mesenteric arteries, and others that are enervated with sympathetic nerves or are proximate one or more sympathetic nerve ganglia. Such a catheter may also be laparoscopically placed in one or more of the above-identified blood vessels, or another luminal tissue without departing from the scope of the present disclosure.

The therapeutic device 50 described herein is configured to deliver stimulation to the blood vessel or other luminal tissue. The amplitude, frequency, pulse width, and/or duration of the stimulation can be selected and/or modified to ensure stimulation of the target nerves of the periluminal tissue (e.g., unmyelinated nerve fibers) without damaging the luminal tissue or the nerves within or surrounding the luminal tissue or causing excess vasoconstriction about the therapeutic device (e.g., inhibiting the movement of the therapeutic device within the luminal tissue).

As noted above, the therapeutic device 50 is coupled to a therapy source 24 and a stimulation source 24a, although it is envisioned that the therapy source 24 and the stimulation source 24a may be the same and capable of generating therapy and neurostimulation,.

In accordance with aspects of the present disclosure, the therapeutic device may be navigated within the vessels or luminal tissue in one configuration (e.g., a linear configuration) and once located at a desired location, deployed, or otherwise actuated to achieve a second configuration.

As noted herein the application of neurostimulation may achieve one of a number of physiological responses including a change in systolic blood pressure, a change in mean arterial blood pressure, a change in vessel stiffness, a change in pulse wave velocity, a change in vessel stiffness, vasoconstriction, and others.

As described hereinabove, it is envisioned that the physiological responses to the application of neurostimulation can be monitored by a control algorithm 44 stored on the computer 22, with the location and results of the application of neurostimulation stored in the memory 32. As noted, the observed post therapy and intra-procedural physiological responses can be compared to the pre-procedural responses to assess the efficacy of the therapy, determine if more therapy is required, and when sufficient therapy has been applied to achieve the desired ablation/denervation.

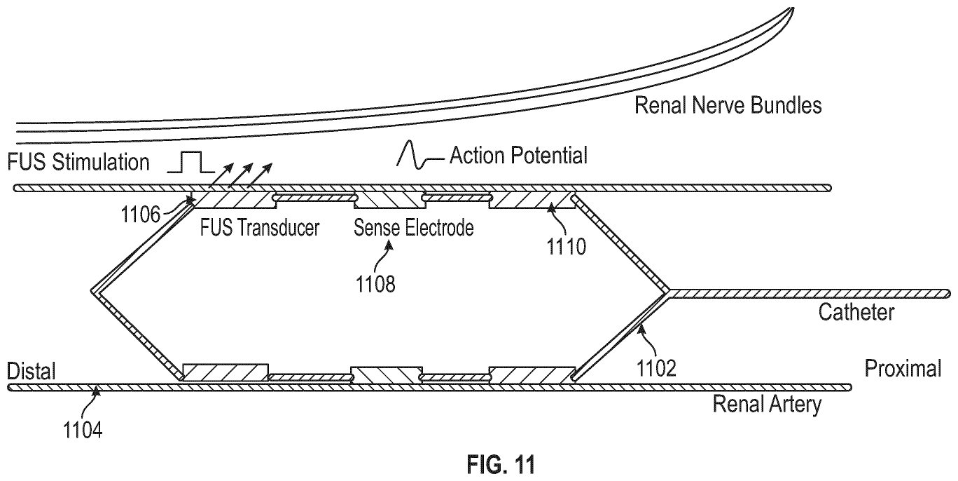

A further aspect of the disclosure is directed to the device depicted in FIG. 11. In FIG. 11, a catheter 1102 is depicted positioned within a blood vessel 1104. The catheter 1102 may be a balloon catheter or may be a frame expandable catheter that allows the blood to continue to flow through the blood vessel substantially unimpeded even when expanded. The catheter 1102 includes a first FUS transducer 1106 formed on a distal portion of the catheter 1102. Proximal of the first FUS transducer 1106 is a sensing electrode 1108 that is configured to detect the electrically evoked compound action potential (ECAP) that is triggered in the nerves (e.g., sympathetic nerves) as a result of the FUS stimulation energy transmitted by the first FUS transducer 1106. Proximal of the sensing electrode is a second FUS transducer 1110 configured to apply FUS therapeutic energy to the nerves to achieve a denervation. As noted above by altering the parameters of the FUS energy, FUS can be utilized for both stimulation and denervation.

In accordance with one aspect of the disclosure, the catheter 1102 is placed in a blood vessel associated with a disease state that benefits from denervation (e.g., placement in the renal or hepatic artery to produce a reduction in hypertension). The first FUS transducer 1106 generates a stimulation pulse that is directed at the walls of the blood vessel. As noted above, this is a mechanical pulse, which triggers an electrical response from the nerves (e.g., ECAP). This response is detected by the sense electrode 1108. The stimulation may be applied in any of the sequential, alternating, or simultaneous session patterns discussed herein. Regardless of the stimulation session pattern employed, the second FUS transducer 1110 is employed to apply HIFU therapeutic energy at the nerves. In order to achieve a denervation of the nerves being stimulated, as described herein above.

By placing the second FUS transducer 1110 proximal of the first FUS transducer 1106, a successful denervation of the afferent nerves can be sensed by the sensing electrode once the nerve is denervated. FUS stimulation and denervation has at least one advantage over the use of electrical modalities such as RF in that the actual ECAP of the nerves can be sensed, not just the effects of the stimulation. This is due in large part to the modality differences of FUS stimulation and denervation as compared to RF stimulation and denervation. In RF stimulation and denervation, and ECAP that might be triggered by the stimulation is substantially lost in the interference caused by the stimulation and denervation signals. Another way to consider this is that the signal being sought the ECAP is lost in the noise created by both the RF stimulation and the RF denervation signals. FUS being substantially mechanical in nature, at least as the energy is applied to the blood vessel wall and therewith the nerves in and around the blood vessel, generates no similar noise and as such the sense electrode 1108 is able to detect the ECAP caused by the FUS stimulation energy whether the FUS denervation energy is being applied by the second FUS transducer 1110 or not. When no ECAP or a substantial reduction in the ECAP is sensed by the sense electrode 1108, the clinician can have confidence that an end point for the therapy has been achieved and that the denervation has been successful. Further one or more applications 34, may utilize the lack of ECAP as an endpoint for the procedure signaling the therapy source 24 and the stimulation source 24a to cease operation, and to generate a display on the user interface 28 alerting the clinician to the sensed end of the denervation procedure and the success of the therapy.

In further aspect of the disclosure, by switching the orientation or the first FUS transducer 1106 and the second FUS transducer 1110. By placing the second FUS transducer 1110 is distal of sense electrode and the first FUS transducer, changes in ECAP of the efferent nerves can be sensed by the sense electrode 1108. Either or both orientations and placement of the first FUS transducer 1106 and the second FUS transducer 1110 relative to the sense electrode 1108 may be employed without departing from the scope of the disclosure to ensure successful denervation of both the efferent and afferent aspects of the nerve.

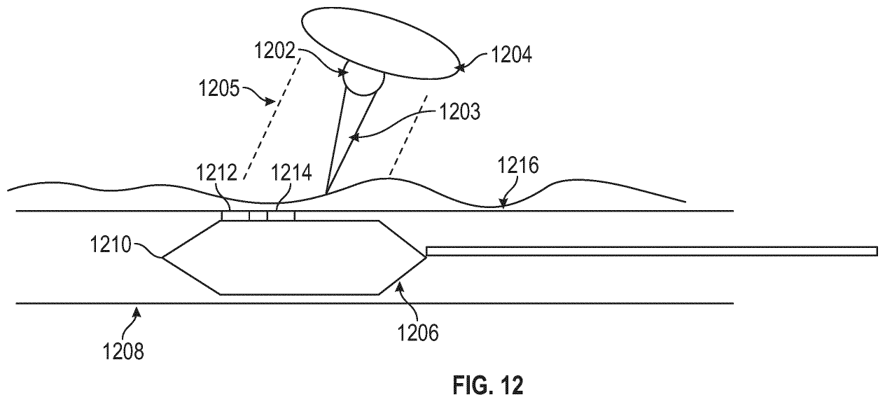

Yet a further aspect of the disclosure is depicted in FIG. 12 in which an external FUS transducer 1202 is depicted, in combination with an imaging ultrasound transducer 1204. A catheter 1206 is depicted within a blood vessel 1208. The catheter includes an expanded section 1210 which may be a balloon or an expandable structure allowing blood flow through the blood vessel 1208. Formed on the expandable section 1210 is an FUS stimulation transducer 1212 and a sense electrode 1214.

In practice, the catheter 1206 is navigated within the blood vessel (e.g., the hepatic or renal arteries). The imaging ultrasound transducer 1204 has an imaging field 1205 that captures images of the catheter 1206 and the blood vessels surrounding the catheter 1206 during placement. One or more of the FUS transducer 1212 or the sense electrode 1214 may include echogenic material rendering it highly visible under ultrasound imaging. Once placed within the blood vessel 1208, the FUS stimulation transducer 1212 can impart energy on the blood vessel wall and the nerves 1216 in or around the blood vessel. The FUS stimulation energy, received by the nerve 1216 elicits an ECAP response that can be sensed by the sense electrode 1214 and viewed within the captured images as vasoconstriction. As described above, the nervous response is indicative of the energy from the FUS stimulation transducer 1212 is impacting the nerves 1216 and that the catheter 1206 is properly placed. The external FUS transducer 1202 can then be engaged to produce a HIFU signal in field 1203, as described above, to denervate the nerve fibers 1216. As in the example of FIG. 11, the external FUS transducer 1202 has may be focused at a point along the blood vessel 1208, and particularly the nerve 1216 proximal of the sense electrode 1214. By aiming the HIFU energy of the external FUS transducer at or proximal to the sense electrode 1214, upon successful denervation of the nerve 1216 any stimulation applied by the FUS transducer 1212 to the afferent nerves will be incapable of evoking any ECAP as a result of the stimulation and/or vasoconstriction of the surrounding blood vessels will be reduced and/or eliminated. Further, at described above, aiming of the HIFU energy of the external FUS transducer 1202 at a point distal of the sense electrode 1214, while the FUS stimulation transducer 1212 is located proximal of the sense electrode 1214, reductions in ECAP of efferent nerves caused by the stimulation energy can be detected and vasoconstriction of the surrounding blood vessels can be visualized. Either or both orientations of the placement of the FUS stimulation transducer 1212 and the aiming of the HIFU energy from the external FUS transducer 1202 may be employed without departing from the scope of the disclosure to ensure successful denervation of both the efferent and afferent aspects of the nerve 1216. Accordingly, whether employing a sequential session, an alternating session, or a simultaneous session, both an end point for the therapy, and a determination of a successful therapy can be ascertained.

A further aspect of the disclosure utilizes the sense electrode 1214 not just for the purposes of sensing ECAP, but also for application of radio frequency (RF) ablation energy to denervate the fibers of the nerves 1216. In such an application the FUS stimulation transducer 1212 is employed as described herein above to stimulate the nerves 1216. This stimulation can be sensed by the sense electrode 1214, as described above, and further, the same sense electrode 1214 may also be configured to deliver RF ablation energy to denervate the nerves 1216. As will be appreciated, the 1206 may include multiple FUS stimulation transducers 1212 and sense electrodes 1214 (configured also to deliver RF ablation energy) such that the stimulation of both afferent and efferent nerves 1216 may be detected and denervation thereof detected.

In yet a further embodiment, the sense electrodes 1214 are just that, but the FUS stimulation transducer 1212 is also configured to deliver RF denervation energy to the nerves 1216. As it understood RF ablation energy is typically delivered at between 400 and 500 kHz, whereas focused ultrasound may be delivered at between 3-5 MHz. Accordingly, the stimulation sources 24a is configured to cause the FUS stimulation transducer 1212 to deliver FUS stimulation energy, and the therapy source 24 may be an RF therapy source configured to deliver RF energy to the nerves 1216 via the FUS stimulation transducer 1212. Again, multiple FUS stimulation transducers 1212 may be employed to stimulate both the afferent and the efferent nerves 1216 and to ensure denervation thereof, as described elsewhere herein.

Returning to FIGS. 13A-13D, as described herein above, it is envisioned that vessel contraction (e.g., for example, vasoconstriction) may be utilized as a modality to identify a physiological response to stimulation. As described hereinabove, stimulation, such as, for example, neurostimulation, of afferent and efferent nerves effectuates a physiological response within the blood vessels 606 or the luminal tissue surrounding the blood vessels, which in embodiments may be vasoconstriction. Vasoconstriction induced by neurostimulation may be local to or may radiate outward from the location where neurostimulation has been applied. In this manner, where neurostimulation has been applied adjacent to a primary bifurcation of the blood vessels, vasoconstriction can be imaged and identified at the location where the neurostimulation has been applied in addition to blood vessels distal of the primary bifurcation (FIG. 13B). As can be appreciated, identifying vasoconstriction within the blood vessels distal of the primary bifurcation can be utilized to assess the efficacy of denervation therapy applied to the afferent and efferent nerves.

It is envisioned that the blood vessels adjacent to or distal to the location where neurostimulation is to be applied (FIG. 13A) and/or has been applied (FIG. 13B) may be imaged using any suitable imaging modality, such as for example, ultrasound, CT, CBCT, and fluoroscopy, and may be imaged from within the blood vessels or external to the blood vessels without departing from the scope of the disclosure. In embodiments, the blood vessels may be imaged using ultrasound emitted from the therapeutic device 50, the imaging ultrasound transducer 1204, the catheter 600, or the HIFU transducer 502. As described hereinabove, the ultrasound device may split the ultrasound array into two or more portions for delivering FUS stimulation energy, imaging, delivering HIFU therapy, and/or delivering RF ablation energy. In embodiments, one or more of stimulation, imaging, HIFU therapy, or RF ablation energy may be delivered using the same or different devices disposed within or external to the target blood vessels. It is contemplated that any suitable means, including those described hereinabove, for stimulating nerves within the blood vessels or luminal tissue surrounding the blood vessels may be utilized without departing from the scope of the disclosure.

In operation, before neurostimulation or therapy has been applied to the blood vessels, the target blood vessels 1300 are imaged using ultrasound either internally, externally, or combinations thereof (FIG. 13A). Thereafter, neurostimulation is applied to the target sympathetic nerves and the target blood vessels are once again imaged using ultrasound (13B). The ultrasound images captured during or after the application of neurostimulation are compared to the ultrasound images captured before neurostimulation to identify constricted blood vessels 1302 at and/or distal to the location where neurostimulation was applied. As can be appreciated, the application of neurostimulation and ultrasound imaging may be performed as many times as necessary at the same or different locations to identify sympathetic nerves that are candidates for denervation. With candidate tissue identified, therapy is applied to the candidate tissue to denervate the nerves 1216 (FIG. 12). After applying therapy, the target blood vessels 1300 are again imaged using ultrasound (FIG. 13C). Thereafter, neurostimulation is again applied to the target sympathetic nerves and the target blood vessels 1300 are once again imaged using ultrasound (FIG. 13C). The ultrasound images captured during or after the application of neurostimulation following therapy are compared to the ultrasound images captured before neurostimulation but after therapy, or in embodiments, to the ultrasound images captured during or after the application of neurostimulation but before therapy, to identify the presence, or absence, of vasoconstriction (FIG. 13D). As can be appreciated, the above described process may be performed as many times as necessary and in any order without departing from the scope of the disclosure.

Although described generally hereinabove, it is envisioned that the memory 32 may include any non-transitory computer-readable storage media for storing data and/or software including instructions that are executable by the processor 30 and which control the operation of the workstation 20 and, in some embodiments, may also control the operation of the therapeutic device 50. In an embodiment, memory 32 may include one or more storage devices such as solid-state storage devices, e.g., flash memory chips. Alternatively, or in addition to the one or more solid-state storage devices, the memory 32 may include one or more mass storage devices connected to the processor 30 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 30. That is, computer readable storage media may include non-transitory, volatile, and non-volatile, removable, and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 20.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

The invention may be further described by reference to the following numbered paragraphs:
1. A method of performing a therapeutic procedure, comprising:
applying a first focused ultrasound (FUS) stimulation energy to tissue in or proximate a blood vessel wall;
observing a patient parameter in response to the first stimulation energy;
applying a therapy to the blood vessel wall;
applying a second FUS stimulation energy to the blood vessel wall;
observing the patient parameter in response to the second FUS stimulation energy; and
determining whether the therapy has been successful based on the observed patient parameter in response to the second FUS stimulation energy.
2. The method of paragraph 1, wherein the patient parameter comprises at least one of blood pressure, pulse wave velocity (PWV) within a blood vessel associated with the blood vessel wall, blood vessel diameter, or electrically evoked compound action potential (ECAP) from a nerve in or proximate the blood vessel wall.
3. The method of paragraph 2, wherein the first FUS stimulation energy and the second FUS stimulation energy are applied to sympathetic nerves in or proximate the blood vessel wall.
4. The method of paragraph 2, wherein the blood vessel is a hepatic or a renal artery.
5. The method of any of the preceding paragraphs, further comprising determining whether the at least one of blood pressure, PWV, or ECAP observed following the application of the second FUS stimulation energy is less than the at least one of blood pressure, PWV, or ECAP observed following the first FUS stimulation energy.
6. The method of any of the preceding paragraphs, further comprising determining whether a change in the at least one of blood pressure, PWV, blood vessel diameter, or ECAP between the first FUS stimulation energy and the second FUS stimulation is greater than a threshold, wherein if the change is greater than the threshold the therapy is determined to be successful.
7. The method of any of the preceding paragraphs, further comprising navigating a therapy device to a location within an artery of the patient.
8. The method of any of the preceding paragraphs, further comprising laparoscopically placing a FUS energy delivery device extravascularly proximate the blood vessel wall.
9. The method of any of the preceding paragraphs, further comprising applying FUS energy from outside a body of a patient to a nerve in or proximate the blood vessel wall.
10. The method of any of the preceding claims, further comprising imaging the blood vessel to determine a PWV within the blood vessel or a diameter of the blood vessel, wherein a PWV in excess of a threshold is indicative a patient that is a candidate for a denervation procedure or a blood vessel diameter that is less than a threshold is indicative of a patient that is a candidate for a denervation procedure.
11. The method of paragraph 2, wherein the first FUS stimulation energy and the second FUS stimulation energy are part of a sequential session, an alternating session, or a simultaneous session.
12. A system for denervation of nerves of a blood vessel comprising:
a catheter configured for navigation within a blood vessel of a patient;
at least one energy element formed on a distal portion of the catheter;
a sensor configured to measure one or more patient parameters operably connected to the catheter;
a focused ultrasound (FUS) stimulation energy source operably connected to the catheter;
a therapeutic energy source configured to apply therapy to the blood vessel; and
a processing means configured to:
   cause the FUS stimulation energy source to generate a first FUS stimulation energy for application to a blood vessel wall via the at least one energy element;
   cause the sensor to sense a first change in a patient parameter as a result of the application of the first FUS stimulation energy to the blood vessel wall;
   cause the therapeutic energy source to generate a therapeutic energy for application to or proximate the blood vessel;
   cause the FUS stimulation energy source to generate a second FUS stimulation energy for application to the blood vessel wall via the at least one energy elements;
   cause the sensor to sense a second change in the patient parameter as result of the application of the second FUS stimulation energy to the blood vessel wall; and
   determine whether the therapy has been successful based on the sensed second change in the patient parameter in response to the second FUS stimulation energy.
13. The system of paragraph 12, wherein the patient parameter comprises at least one of blood pressure, pulse wave velocity (PWV) within the blood vessel, blood vessel diameter, or electrically evoked compound action potential (ECAP) from a nerve in or proximate the blood vessel wall.
14. The system of paragraph 12, wherein the first FUS stimulation energy and the second FUS stimulation energy are applied to sympathetic nerves in or proximate the blood vessel wall.
15 The system of paragraph 12, wherein the blood vessel is a hepatic or a renal artery.
16. The system of any of the preceding paragraphs, wherein the processing means is further configured to determine whether at least one of blood pressure, PWV, or ECAP observed following the application of the second FUS stimulation energy is less than at least one of the blood pressure, PWV, or ECAP observed following the first FUS stimulation energy
17. The system of any of the preceding paragraphs, wherein the processing means is further configured to determine whether a change in blood pressure, PWV, blood vessel diameter, or ECAP between the first FUS stimulation energy and the second FUS stimulation is greater than a threshold, wherein if the change is greater than the threshold the therapy is successful.
18. The system of paragraph 13, wherein the generated first ultrasound stimulation energy and the generated second ultrasound stimulation energy are part of a sequential session, an alternating session, or a simultaneous session.
19. The system of paragraph 13, wherein the therapeutic energy source is a focused ultrasound energy source in communication with one of the at least one energy element formed on a distal portion of the catheter.
20. The system of any of the preceding paragraphs, further comprising an external focused ultrasound transducer operably connected to the therapeutic energy source and configured to apply high intensity focused ultrasound energy to or proximate to the blood vessel.

Further disclosed herein is the subject-matter of the following clauses:
1. A method (800) of operating a surgical system (10), comprising:
applying a first focused ultrasound (FUS) stimulation energy to tissue in or proximate a blood vessel wall;
observing a patient parameter in response to the first stimulation energy;
applying a therapy to the blood vessel wall;
applying a second FUS stimulation energy to the blood vessel wall;
observing the patient parameter in response to the second FUS stimulation energy; and
determining whether the therapy has been successful based on the observed patient parameter in response to the second FUS stimulation energy.
2. The method of clause 1, wherein the patient parameter comprises at least one of blood pressure, pulse wave velocity (PWV) within a blood vessel associated with the blood vessel wall, blood vessel diameter, or electrically evoked compound action potential (ECAP) from a nerve in or proximate the blood vessel wall.
3. The method of clause 2, wherein the first FUS stimulation energy and the second FUS stimulation energy are applied to sympathetic nerves in or proximate the blood vessel wall; or
wherein the blood vessel is a hepatic or a renal artery.
4. The method of any of the preceding clauses, further comprising determining whether the at least one of blood pressure, PWV, or ECAP observed following the application of the second FUS stimulation energy is less than the at least one of blood pressure, PWV, or ECAP observed following the first FUS stimulation energy; or
further comprising determining whether a change in the at least one of blood pressure, PWV, blood vessel diameter, or ECAP between the first FUS stimulation energy and the second FUS stimulation is greater than a threshold, wherein if the change is greater than the threshold the therapy is determined to be successful.
5. The method of any of the preceding clauses, further comprising navigating a therapy device (50) to a location within an artery of the patient; or
further comprising laparoscopically placing a FUS energy delivery device (502) extravascularly proximate the blood vessel wall.
6. The method of any of the preceding clauses, further comprising applying FUS energy from outside a body of a patient to a nerve in or proximate the blood vessel wall.
7. The method of any of the preceding clauses, further comprising imaging the blood vessel to determine a PWV within the blood vessel or to determine a diameter of the blood vessel, wherein a PWV in excess of a threshold is indicative a patient that is a candidate for a denervation procedure or a blood vessel diameter that is less than a threshold is indicative of a patient that is a candidate for a denervation procedure.
8 The method of clause 2, wherein the first FUS stimulation energy and the second FUS stimulation energy are part of a sequential session, an alternating session, or a simultaneous session.
9. A system (10) for denervation of nerves of a blood vessel comprising:
a catheter (50) configured for navigation within a blood vessel of a patient;
at least one energy element (56) formed on a distal portion of the catheter;
a sensor (60) configured to measure one or more patient parameters operably connected to the catheter;
a focused ultrasound (FUS) stimulation energy source (24a) operably connected to the
a therapeutic energy source (24) configured to apply therapy to the blood vessel; and
a processing means (20) configured to:
   cause the FUS stimulation energy source to generate a first FUS stimulation energy for application to a blood vessel wall via the at least one energy element;
   cause the sensor to sense a first change in a patient parameter as a result of the application of the first FUS stimulation energy to the blood vessel wall;
   cause the therapeutic energy source to generate a therapeutic energy for application to or proximate the blood vessel;
   cause the FUS stimulation energy source to generate a second FUS stimulation energy for application to the blood vessel wall via the at least one energy elements;
   cause the sensor to sense a second change in the patient parameter as result of the application of the second FUS stimulation energy to the blood vessel wall; and
   determine whether the therapy has been successful based on the sensed second change in the patient parameter in response to the second FUS stimulation energy.
10. The system of clause 9, wherein the patient parameter comprises at least one of blood pressure, pulse wave velocity (PWV) within the blood vessel, blood vessel diameter, or electrically evoked compound action potential (ECAP) from a nerve in or proximate the blood vessel wall.
11. The system of clause 9, wherein the first FUS stimulation energy and the second FUS stimulation energy are applied to sympathetic nerves in or proximate the blood vessel wall; or
wherein the blood vessel is a hepatic or a renal artery.
12. The system of any of the preceding clauses, wherein the processing means is further configured to determine whether at least one of blood pressure, PWV, or ECAP observed following the application of the second FUS stimulation energy is less than at least one of the blood pressure, PWV, or ECAP observed following the first FUS stimulation energy.
13. The system of any of the preceding clauses, wherein the processing means is further configured to determine whether a change in blood pressure, PWV, or ECAP between the first FUS stimulation energy and the second FUS stimulation is greater than a threshold, wherein if the change is greater than the threshold the therapy is successful.
14. The system of clause 9, wherein the generated first ultrasound stimulation energy and the generated second ultrasound stimulation energy are part of a sequential session, an alternating session, or a simultaneous session; or
wherein the therapeutic energy source is a focused ultrasound energy source in communication with one of the at least one energy element formed on a distal portion of the catheter.
15. The system of clause 9, further comprising an external focused ultrasound transducer operably connected to the therapeutic energy source and configured to apply high intensity focused ultrasound energy to or proximate to the blood vessel.

1-15. (canceled)

16. A method of performing a therapeutic procedure, comprising:
applying a first focused ultrasound (FUS) stimulation energy to tissue in or proximate a blood vessel wall;
observing a patient parameter in response to the first stimulation energy;
applying a therapy to the blood vessel wall;
applying a second FUS stimulation energy to the blood vessel wall;
observing the patient parameter in response to the second FUS stimulation energy; and
determining whether the therapy has been successful based on the observed patient parameter in response to the second FUS stimulation energy.

17. The method of claim 16, wherein the patient parameter comprises at least one of blood pressure, pulse wave velocity (PWV) within a blood vessel associated with the blood vessel wall, or electrically evoked compound action potential (ECAP) from a nerve in or proximate the blood vessel wall.

18. The method of claim 17, wherein the first FUS stimulation energy and the second FUS stimulation energy are applied to sympathetic nerves in or proximate the blood vessel wall.

19. The method of claim 17, wherein the blood vessel is a hepatic or a renal artery.

20. The method of claim 17, further comprising determining whether the at least one of blood pressure, PWV, or ECAP observed following the application of the second FUS stimulation energy is less than the at least one of blood pressure, PWV, or ECAP observed following the first FUS stimulation energy.

21. The method of claim 17, further comprising determining whether a change in the at least one of blood pressure, PWV, or ECAP between the first FUS stimulation energy and the second FUS stimulation is greater than a threshold, wherein if the change is greater than the threshold the therapy is determined to be successful.

22. The method of claim 17, further comprising navigating a therapy device to a location within an artery of the patient.

23. The method of claim 17, further comprising laparoscopically placing a FUS energy delivery device extravascularly proximate the blood vessel wall.

24. The method of claim 17, further comprising applying FUS energy from outside a body of a patient to a nerve in or proximate the blood vessel wall.

25. The method of claim 17, further comprising imaging the blood vessel to determine a PWV within the blood vessel, wherein a PWV in excess of a threshold is indicative a patient that is a candidate for a denervation procedure.

26. The method of claim 17, wherein the first FUS stimulation energy and the second FUS stimulation energy are part of a sequential session, an alternating session, or a simultaneous session.

27. A system for denervation of nerves of a blood vessel comprising:
a catheter configured for navigation within a blood vessel of a patient;
at least one energy element formed on a distal portion of the catheter;
a sensor configured to measure one or more patient parameters operably connected to the catheter;
a focused ultrasound (FUS) stimulation energy source operably connected to the catheter;
a therapeutic energy source configured to apply therapy to the blood vessel; and
a computing device including a memory and a processor and storing thereon instructions that when executed cause the computing device to:
cause the FUS stimulation energy source to generate a first FUS stimulation energy for application to a blood vessel wall via the at least one energy element;
cause the sensor to sense a first change in a patient parameter as a result of the application of the first FUS stimulation energy to the blood vessel wall;
cause the therapeutic energy source to generate a therapeutic energy for application to or proximate the blood vessel;
cause the FUS stimulation energy source to generate a second FUS stimulation energy for application to the blood vessel wall via the at least one energy elements;
cause the sensor to sense a second change in the patient parameter as result of the application of the second FUS stimulation energy to the blood vessel wall; and
determine whether the therapy has been successful based on the sensed second change in the patient parameter in response to the second FUS stimulation energy.

28. The system of claim 27, wherein the patient parameter comprises at least one of blood pressure, pulse wave velocity (PWV) within the blood vessel or electrically evoked compound action potential (ECAP) from a nerve in or proximate the blood vessel wall.

29. The system of claim 27, wherein the first FUS stimulation energy and the second FUS stimulation energy are applied to sympathetic nerves in or proximate the blood vessel wall.

30. The system of claim 27, wherein the blood vessel is a hepatic or a renal artery.

31. The system of claim 28, wherein the memory stores thereon instructions that when executed by the processor cause the computing device to determine whether at least one of blood pressure, PWV, or ECAP observed following the application of the second FUS stimulation energy is less than at least one of the blood pressure, PWV, or ECAP observed following the first FUS stimulation energy.

32. The system of claim 28, wherein the memory stores thereon instructions that when executed by the processor cause the computing device to determine whether a change in blood pressure, PWV, or ECAP between the first FUS stimulation energy and the second FUS stimulation is greater than a threshold, wherein if the change is greater than the threshold the therapy is successful.

33. The system of claim 28, wherein the generated first ultrasound stimulation energy and the generated second ultrasound stimulation energy are part of a sequential session, an alternating session, or a simultaneous session.

34. The system of claim 28, wherein the therapeutic energy source is a focused ultrasound energy source in communication with one of the at least one energy element formed on a distal portion of the catheter.

35. The system of claim 28, further comprising an external focused ultrasound transducer operably connected to the therapeutic energy source and configured to apply high intensity focused ultrasound energy to or proximate to the blood vessel.
